(19) United States
(12) Patent Application Publication
(10) Pub. No.: US 2026/0235438 A1
(43) Pub. Date: Aug. 13, 2026

(54) DETECTION DEVICE

(71) Applicant: Japan Display Inc., Tokyo (JP)

(72) Inventor: Gen KOIDE, Tokyo (JP)

(21) Appl. No.: 19/639,679

(22) Filed: Apr. 6, 2026

Related U.S. Application Data
(63) Continuation of application No. PCT/JP2024/034061, filed on Sep. 25, 2024.

(30) Foreign Application Priority Data
Oct. 13, 2023 (JP) 2023-177282

(51) Int. Cl. G01J 1/44; A61B 5/1455; G01R 19/10; G06V 10/141; G06V 40/13; G06V 40/145

(52) U.S. Cl. CPC G01J 1/44; G01R 19/10; A61B 5/14551; G01J 2001/446; G06V 10/141; G06V 40/1318; G06V 40/145

(57) ABSTRACT

According to an aspect, a detection device includes: an optical sensor; a light source configured to emit light to the optical sensor; and a detection circuit including an integrating circuit coupled to the optical sensor. The integrating circuit is provided so as to be changeable in gain corresponding to a slope of output voltage characteristics representing a relation between a light intensity irradiating the optical sensor and an output voltage of the integrating circuit. The optical sensor is configured to measure the light intensity both when the light source is off and when the light source is on. The integrating circuit is configured to be reduced in the gain as a light intensity of external light measured with the light source off increases.

### CROSS-REFERENCE TO RELATED APPLICATION

This application claims the benefit of priority from Japanese Patent Application No. 2023-177282 filed on Oct. 13, 2023 and International Patent Application No. PCT/JP2024/034061 filed on Sep. 25, 2024, the entire contents of which are incorporated herein by reference.

### BACKGROUND

### 1. Technical Field

What is disclosed herein relates to a detection device.

### 2. Description of the Related Art

Optical sensors capable of detecting fingerprint patterns and vein patterns are known (for example, Japanese Patent Application Laid-open Publication No. 2009-032005). Such an optical sensor includes a plurality of photodiodes each formed with an organic semiconductor material as an active layer.

In such a detection device, the intensity of light incident on the optical sensor varies depending, for example, on measurement conditions such as the presence or absence of external light, the light intensity of the external light and light from a light source, the measurement portion of an object to be detected, and the distance between the object to be detected and the light source. Therefore, the detection accuracy of the detection device may be lowered.

For the foregoing reasons, there is a need for a detection device capable of improving the detection accuracy.

### SUMMARY

According to an aspect, a detection device includes: an optical sensor; a light source configured to emit light to the optical sensor; and a detection circuit including an integrating circuit coupled to the optical sensor. The integrating circuit is provided so as to be changeable in gain corresponding to a slope of output voltage characteristics representing a relation between a light intensity irradiating the optical sensor and an output voltage of the integrating circuit. The optical sensor is configured to measure the light intensity both when the light source is off and when the light source is on. The integrating circuit is configured to be reduced in the gain as a light intensity of external light measured with the light source off increases.

### BRIEF DESCRIPTION OF THE DRAWINGS

FIG. 1 is a plan view schematically illustrating a detection device according to an embodiment;

FIG. 2 is a sectional view taken along II-II′ in FIG. 1;

FIG. 3 is a block diagram illustrating a configuration example of the detection device according to the embodiment;

FIG. 4 is a circuit diagram illustrating the configuration example of the detection device according to the embodiment;

FIG. 5 is a timing diagram for explaining a detection method of the detection device according to the embodiment;

FIG. 6 is an explanatory diagram illustrating a relation between an integrated light intensity and an output voltage of an integrating circuit in a detection device according to a comparative example;

FIG. 7 is a flowchart illustrating a method for adjusting output voltage characteristics indicating the relation between the integrated light intensity and the output voltage of the integrating circuit in the detection device according to the embodiment;

FIG. 8 is a graph for explaining a method for calculating a virtual photocurrent caused by a light source at Step ST2 in FIG. 7;

FIG. 9 is an explanatory diagram for explaining a method for calculating an exposure period at Step ST3 in FIG. 7;

FIG. 10 is a graph for explaining a method for adjusting a gain and an offset amount at Steps ST4 and ST5 in FIG. 7;

FIG. 11 is a graph for explaining a waveform and a measurement range of each output voltage in FIG. 10;

FIG. 12 is a graph for explaining a method for calculating a drive current to be supplied to the light source;

FIG. 13 is a schematic view illustrating an exemplary external appearance when a finger accommodated inside a detection device according to a modification of the embodiment is viewed from a lateral side of a housing; and

FIG. 14 is a sectional view taken along XIV-XIV′ in FIG. 13.

### DETAILED DESCRIPTION

The following describes a mode (embodiment) for carrying out the present disclosure in detail with reference to the drawings. The present disclosure is not limited to the description of the embodiment given below. Components described below include those easily conceivable by those skilled in the art or those substantially identical thereto. In addition, the components described below can be combined as appropriate. What is disclosed herein is merely an example, and the present disclosure naturally encompasses appropriate modifications easily conceivable by those skilled in the art while maintaining the gist of the present disclosure. To further clarify the description, the drawings may schematically illustrate, for example, widths, thicknesses, and shapes of various parts as compared with actual aspects thereof. However, they are merely examples, and interpretation of the present disclosure is not limited thereto. The same component as that described with reference to an already mentioned drawing is denoted by the same reference numeral through the present disclosure and the drawings, and detailed description thereof may not be repeated where appropriate.

In the present specification and claims, in expressing an aspect of disposing another structure on or above a certain structure, a case of simply expressing “on” includes both a case of disposing the other structure immediately on the certain structure so as to contact the certain structure and a case of disposing the other structure above the certain structure with still another structure interposed therebetween, unless otherwise specified.

### Embodiment

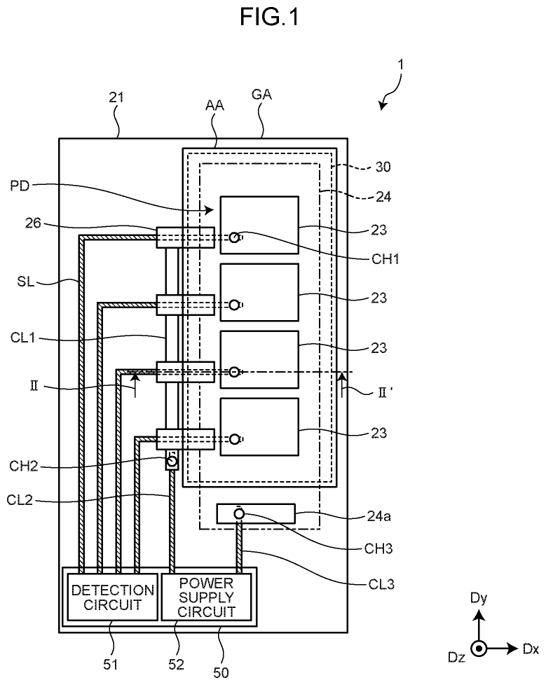

FIG. 1 is a plan view schematically illustrating a detection device according to an embodiment of the present invention. As illustrated in FIG. 1, a detection device 1 includes a substrate 21, a plurality of photodiodes PD (optical sensors), a plurality of signal lines SL, a plurality of shield layers 26, power supply wiring lines CL1, CL2, and CL3, and a control circuit 50.

In the following description, a first direction Dx is one direction in a plane parallel to the substrate 21. A second direction Dy is one direction in the plane parallel to the substrate 21 and is a direction orthogonal to the first direction Dx. The second direction Dy may non-orthogonally intersect the first direction Dx. A third direction Dz is a direction orthogonal to the first direction Dx and the second direction Dy. The third direction Dz is a direction normal to the substrate 21. The term “plan view” refers to a positional relation as viewed from a direction orthogonal to the substrate 21.

The substrate 21 has a detection area AA and a peripheral area GA. The detection area AA is an area provided with the photodiodes PD. The peripheral area GA is an area between the outer perimeter of the detection area AA and the ends of the substrate 21 and is an area not provided with the photodiodes PD. The signal lines SL and the control circuit 50 are provided in the peripheral area GA of the substrate 21.

The detection device 1 includes the photodiodes PD as optical sensor elements. Each of the photodiodes PD outputs an electrical signal in response to light emitted thereto. More specifically, the photodiode PD is an organic photodiode (OPD) including an organic semiconductor. The photodiodes PD are arranged in the second direction Dy in the detection area AA.

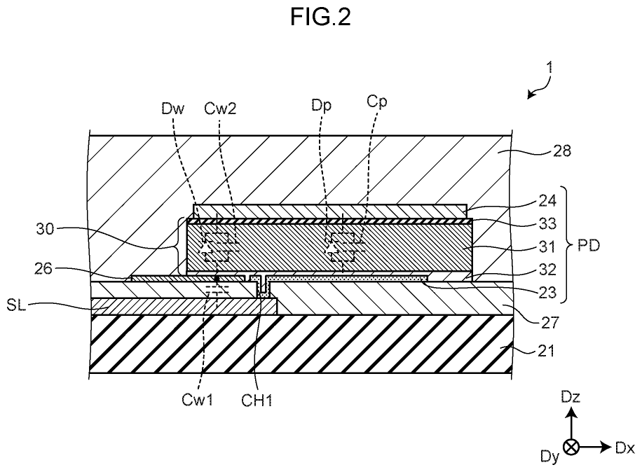

The photodiodes PD each include an organic semiconductor layer 30 (lower buffer layer 32, active layer 31, and upper buffer layer 33 (refer to FIG. 2)), a lower electrode 23 disposed below the organic semiconductor layer 30, and an upper electrode 24 disposed above the upper side of the organic semiconductor layer 30. A plurality of the lower electrodes 23 are provided, one for each of the photodiodes PD, and are arranged in the second direction Dy in the detection area AA. The lower electrodes 23 are arranged apart from one another in the second direction Dy. The organic semiconductor layer 30 and the upper electrode 24 are provided across the photodiodes PD and are provided continuously in the detection area AA. To facilitate viewing of the drawing, FIG. 1 illustrates the organic semiconductor layer 30 and the upper electrode 24 provided on the upper side of the lower electrode 23 with a dashed line and a long dashed double-short dashed line, respectively. The multilayer configuration of the photodiodes PD, the lower electrodes 23, and the upper electrode 24 will be described later with reference to FIG. 2.

One end of each of the signal lines SL is electrically coupled to a corresponding one of the lower electrodes 23 of the photodiodes PD. The other end of each of the signal lines SL is electrically coupled to a detection circuit 51 included in the control circuit 50. In other words, the detection circuit 51 is electrically coupled to the lower electrodes 23 of the photodiodes PD via the signal lines SL.

The shield layers 26 are arranged overlapping the respective signal lines SL in plan view. In more detail, each of the shield layers 26 overlaps a portion of a corresponding one of the signal lines SL extending in the first direction Dx and extends in the first direction Dx along the signal line SL. The shield layers 26 are arranged in the second direction Dy.

The shield layers 26 are coupled to a power supply circuit 52 included in the control circuit 50 via the power supply wiring lines CL1 and CL2 extending in the second direction Dy. More specifically, the power supply wiring line CL1 is provided in the same layer as the shield layers 26 so as to intersect the shield layers 26. The power supply wiring line CL2 is provided in the same layer as the signal lines SL. One end of the power supply wiring line CL2 is electrically coupled to the power supply wiring line CL1 via a contact hole CH2. The other end of the power supply wiring line CL2 is electrically coupled to the power supply circuit 52.

With such a configuration, the power supply circuit 52 supplies a reference voltage VCOM to the shield layers 26 via the power supply wiring lines CL1 and CL2. The reference voltage VCOM is a voltage signal having a fixed predetermined potential. The reference voltage VCOM is, for example, a voltage signal having a potential equal to a first reference potential Vref supplied to the lower electrode 23. The first reference potential Vref is a fixed predetermined potential. The coupling between the shield layers 26 and the power supply circuit 52 may have any configuration, and the arrangement, number, and other factors of the power supply wiring lines CL1 and CL2 can be changed as appropriate.

The upper electrode 24 is provided so as to extend in the second direction Dy across the detection area AA and the peripheral area GA. The upper electrode 24 is electrically coupled to the power supply wiring line CL3 via a contact hole CH3 and a terminal 24a in the peripheral area GA. The power supply wiring line CL3 is coupled to the power supply circuit 52.

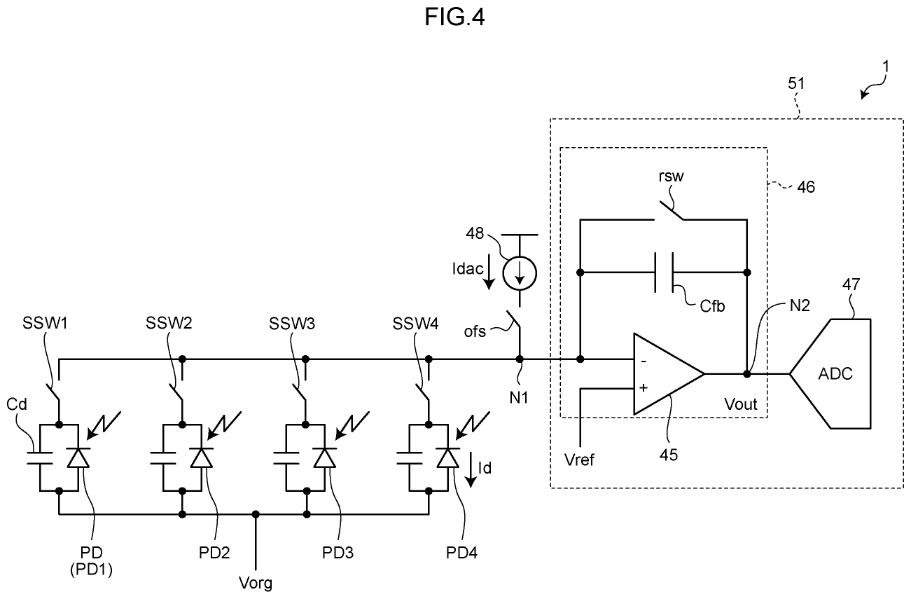

With such a configuration, the upper electrode 24 of the photodiodes PD is coupled to the power supply circuit 52 included in the control circuit 50 via the terminal 24a and the power supply wiring line CL3. The power supply circuit 52 supplies a second reference potential Vorg (refer to FIG. 4) to the upper electrode 24 of the photodiodes PD. The second reference potential Vorg is a fixed predetermined potential.

The control circuit 50 (detection circuit 51 and power supply circuit 52) is located adjacent to the photodiodes PD in the second direction Dy in the peripheral area GA of the substrate 21. The control circuit 50 is a circuit that controls detection operations by supplying control signals to the photodiodes PD. Each of the photodiodes PD outputs, to the detection circuit 51, an electrical signal (photocurrent Id) corresponding to the light emitted thereto. Thereby, the detection device 1 detects information on an object to be detected based on the photocurrents Id from the photodiodes PD.

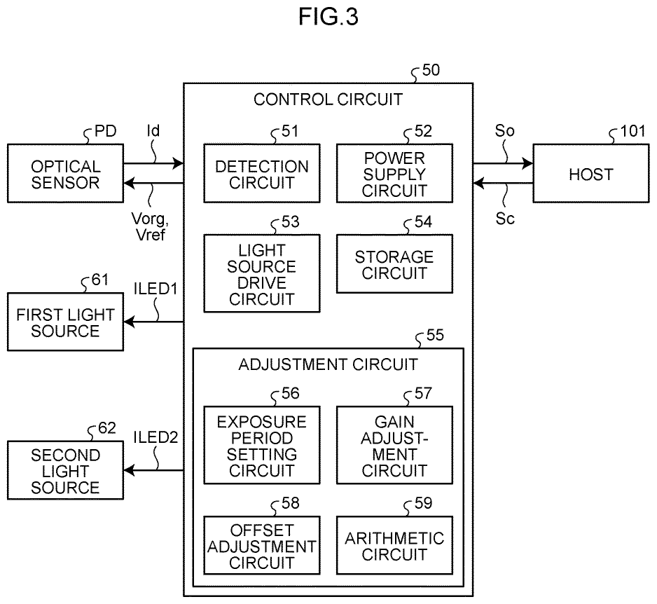

A detailed exemplary configuration of the control circuit 50 and the detection operations of the photodiodes PD will be described later with reference to FIGS. 3 to 12. The control circuit 50 is provided on the same substrate 21 as the photodiodes PD, but is not limited to this configuration. The control circuit 50 may be provided on another control substrate coupled to the substrate 21 via, for example, a flexible printed circuit board or the like. The detection circuit 51 and the power supply circuit 52 included in the control circuit 50 may each be formed as an individual circuit.

Although not illustrated in FIG. 1, the detection device 1 includes a first light source 61 and a second light source 62 (refer to FIG. 3). For example, an inorganic light-emitting diode (LED) or an organic electroluminescent (EL) diode (organic light-emitting diode (OLED)) is used as each of the first and the second light sources 61 and 62. The wavelength of light emitted from the first light source 61 is different from that of light emitted from the second light source 62. For example, the first light source 61 emits near-infrared light or infrared light. The second light source 62 emits green light or red light. The green light has a wavelength of 490 nm to 550 nm, for example. The red light has a wavelength of 640 nm to 770 nm, for example. The infrared light has a wavelength of approximately 2500 nm to approximately 25 μm, for example. The near-infrared light has a wavelength of approximately 770 nm to approximately 2500 nm, for example.

The light emitted from the first and the second light sources 61 and 62 is reflected on a surface of the object to be detected, such as a finger, and enters the photodiodes PD. As a result, the detection device 1 can detect a fingerprint by detecting a shape of asperities on the surface of the finger or the like. Alternatively, the light emitted from the first and the second light sources 61 and 62 may be reflected in the finger or the like, or transmitted through the finger or the like, and enter the photodiodes PD. As a result, the detection device 1 can detect information on a living body in the finger or the like. Examples of the information on the living body include, but are not limited to, pulse waves, pulsation, and a vascular image of the finger or a palm. That is, the detection device 1 may be configured as a fingerprint detection device to detect the fingerprint or a vein detection device to detect a vascular pattern of, for example, veins.

The detection device 1 of the present embodiment can detect an oxygen saturation level in blood (hereinafter referred to as a “blood oxygen saturation level (SpO₂)”) in addition to the pulse waves, the pulsation, and the vascular image as the information on the living body based on the light emitted from the first light source 61 and the light emitted from the second light source 62. Thus, the detection device 1 includes the first and the second light sources 61 and 62, and performs the detection based on the light rays having different wavelengths emitted from these light sources, and thereby can detect the various type of information on the living body. The emission colors of the first and the second light sources 61 and 62 described above are examples, and the present disclosure is not limited by the emission colors of the first and the second light sources 61 and 62.

The following describes a multilayer configuration of the photodiode PD and the shield layer 26. FIG. 2 is a sectional view taken along II-II′ in FIG. 1.

In the following description, a direction from the substrate 21 toward a sealing film 28 in a direction orthogonal to a surface of the substrate 21 is referred to as an “upper side” or simply “above”. A direction from the sealing film 28 toward the substrate 21 is referred to as a “lower side” or simply “below”.

As illustrated in FIG. 2, the substrate 21 is an insulating substrate and is made using, for example, glass or a resin material. The substrate 21 is not limited to having a flat plate shape and may have a curved surface. In this case, the substrate 21 may be made of a film-like resin.

The signal line SL is provided on the substrate 21. The signal line SL is formed, for example, of metal wiring, and is formed of a material having better conductivity than the lower electrode 23 of the photodiode PD. An insulating film 27 is provided on the substrate 21 so as to cover the signal line SL. The insulating film 27 may be an inorganic insulating film or an organic insulating film. The insulating film 27 may be a single layer or a multilayered film.

The photodiode PD is provided on the insulating film 27. In more detail, the photodiode PD includes the lower electrode 23, the lower buffer layer 32, the active layer 31, the upper buffer layer 33, and the upper electrode 24. In the photodiode PD, the lower electrode 23, the lower buffer layer 32, the active layer 31, the upper buffer layer 33, and the upper electrode 24 are stacked in this order in a direction orthogonal to the substrate 21.

The lower electrode 23 is provided on the insulating film 27 and is electrically coupled to the signal line SL via the contact hole CH1 provided in the insulating film 27. The lower electrode 23 is a cathode electrode of the photodiode PD and is formed, for example, of a light-transmitting conductive material such as indium tin oxide (ITO).

The active layer 31 changes in characteristics (for example, voltage-current characteristics and resistance value) depending on light emitted thereto. An organic material is used as a material of the active layer 31. Specifically, the active layer 31 has a bulk heterostructure containing a mixture of a p-type organic semiconductor and an n-type fullerene derivative ((6,6)-phenyl-C61-butyric acid methyl ester (PCBM)) that is an n-type organic semiconductor. As the active layer 31, low-molecular-weight organic materials can be used including, for example, fullerene (C₆₀), phenyl-C₆₁-butyric acid methyl ester (PCBM), copper phthalocyanine (CuPc), fluorinated copper phthalocyanine (F₁₆CuPc), 5,6,11,12-tetraphenyltetracene (rubrene), and perylene diimide (PDI) (derivative of perylene).

The active layer 31 can be formed by a vapor deposition process (dry process) using any of the low-molecular-weight organic materials listed above. In this case, the active layer 31 may be, for example, a multilayered film of CuPc and F₁₆CuPc, or a multilayered film of rubrene and C₆₀. The active layer 31 can also be formed by a coating process (wet process). In this case, the active layer 31 is made using a material obtained by combining any of the above-listed low-molecular-weight organic materials with a high-molecular-weight organic material. As the high-molecular-weight organic material, for example, poly(3-hexylthiophene) (P3HT) and F8-alt-benzothiadiazole (F8BT) can be used. The active layer 31 can be a film made of a mixture of P3HT and PCBM, or a film made of a mixture of F8BT and PDI.

The lower buffer layer 32 is an electron transport layer, and the upper buffer layer 33 is a hole transport layer. The lower buffer layer 32 and the upper buffer layer 33 are provided to facilitate holes and electrons generated in the active layer 31 to reach the lower electrode 23 or the upper electrode 24. Polyethylenimine ethoxylated (PEIE) is used as a material of the electron transport layer. The material of the hole transport layer is a metal oxide layer. For example, tungsten oxide (WO₃) or molybdenum oxide is used as the metal oxide layer.

The materials and the manufacturing methods of the lower buffer layer 32, the active layer 31, and the upper buffer layer 33 are merely exemplary, and other materials and manufacturing methods may be used. For example, each of the lower buffer layer 32 and the upper buffer layer 33 is not limited to a single-layer film, and may be formed as a multilayered film that includes an electron block layer and a hole block layer.

The upper electrode 24 is provided on the upper buffer layer 33. The upper electrode 24 is an anode electrode of the photodiode PD, and is continuously formed over the entire detection area AA. In other words, the upper electrode 24 is continuously provided on the organic photodiodes PD. The upper electrode 24 is formed, for example, of a light-transmitting conductive material such as ITO or indium zinc oxide (IZO).

The sealing film 28 is provided on the upper electrode 24. An inorganic film, such as a silicon nitride film or an aluminum oxide film, or a resin film, such as an acrylic film, is used as the sealing film 28. The sealing film 28 is not limited to a single layer, and may be a multilayered film having two or more layers obtained by combining the inorganic film with the resin film mentioned above. The sealing film 28 well seals the photodiode PD, and thus can reduce moisture entering the photodiode PD from the upper surface side thereof.

The shield layer 26 is provided in the same layer as the lower electrode 23 on the insulating film 27. The shield layer 26 is formed of the same material as the lower electrode 23, for example, a light-transmitting conductive material such as ITO. However, the shield layer 26 is not limited to this material, and may be formed of a material, such as a metal material, different from that of the lower electrode 23.

As described above, the shield layers 26 are supplied with the reference voltage VCOM. The shield layer 26 reduces parasitic capacitance between the upper electrode 24 of the photodiode PD and the signal line SL, and reduces unintended capacitive coupling between the photodiode PD (upper electrode 24) and the signal line SL.

The detection device 1 of the present embodiment may have a configuration without the shield layer 26. While the example has been described where the lower electrode 23 is a cathode electrode and the upper electrode 24 is an anode electrode, the present disclosure is not limited to this example. The lower electrode 23 may be an anode electrode and the upper electrode 24 may be a cathode electrode. In that case, the lower buffer layer 32 may be a hole transport layer, and the upper buffer layer 33 may be an electron transport layer.

The following describes an exemplary detection method of the detection device 1 of the present embodiment. FIG. 3 is a block diagram illustrating a configuration example of the detection device according to the embodiment. As illustrated in FIG. 3, the control circuit 50 includes the detection circuit 51, the power supply circuit 52, a light source drive circuit 53, a storage circuit 54, and an adjustment circuit 55.

The detection circuit 51 is a current detection circuit that measures the photocurrent Id output from the photodiode PD. The detection circuit 51 is configured, for example, with an integrating circuit 46 and an analog-to-digital (A/D) conversion circuit 47 (refer to FIG. 4). The detection circuit 51 measures the photocurrent Id output from the photodiode PD, performs signal processing such as an A/D conversion, and outputs a sensor value So corresponding to the photocurrent Id to a host 101.

The power supply circuit 52 supplies the second reference potential Vorg to the anode of the photodiode PD and also supplies the first reference potential Vref to the cathode of the photodiode PD. In the case of the circuit illustrated in FIG. 4, when a reset switch rsw is on, a reference potential equal to the first reference potential Vref is supplied to the cathode of the photodiode PD from the power supply circuit 52 via an output of an operational amplifier 45. The first reference potential Vref is higher than the second reference potential Vorg. As a result, the photodiode PD is driven into a reverse bias state.

The light source drive circuit 53 supplies a light source control signal LED1 to the first light source 61 and a light source control signal LED2 to the second light source 62. The light source drive circuit 53 thereby controls the lighting and non-lighting of the first and second light sources 61 and 62. The first and the second light sources 61 and 62 emit light to the photodiode PD at light intensities corresponding to the light source control signals LED1 and LED2.

The storage circuit 54 temporarily stores therein the information on the photocurrent Id measured by the detection circuit 51. The storage circuit 54 also stores therein in advance various types of information (coefficients r_th and r_A, margins r_margin1 and r_margin2, and other parameters) used for adjusting output voltage characteristics of the integrating circuit 46 to be described later.

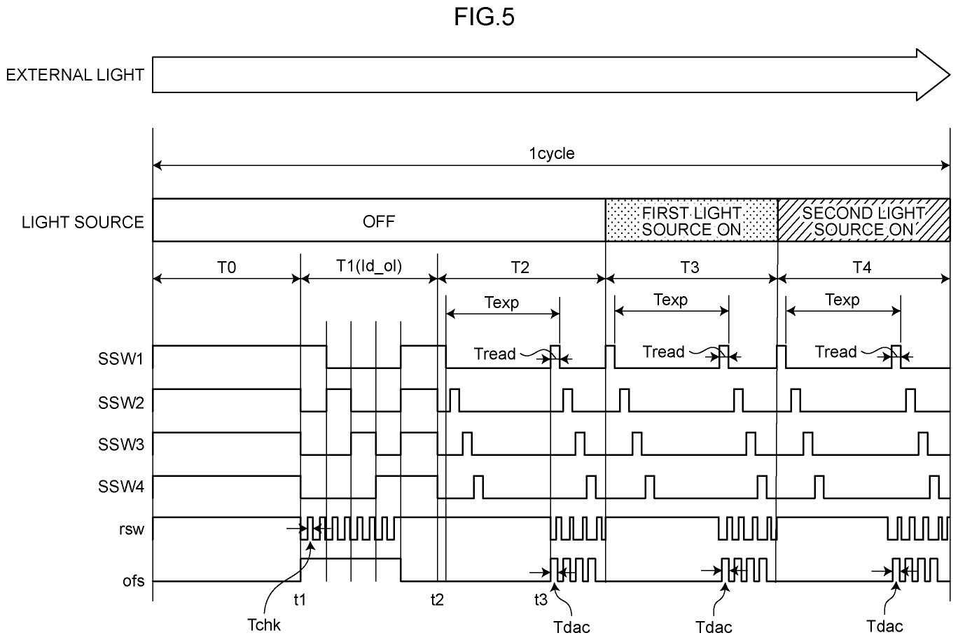

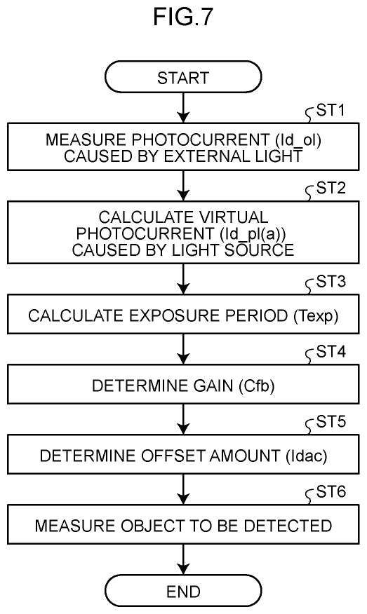

The adjustment circuit 55 includes an exposure period setting circuit 56, a gain adjustment circuit 57, an offset adjustment circuit 58, and an arithmetic circuit 59. The adjustment circuit 55 adjusts an exposure period Texp (refer to FIG. 5) in the detection by the photodiode PD and various parameters, such as the gain and the offset of the integrating circuit 46 included in the detection circuit 51. Operations of the exposure period setting circuit 56, the gain adjustment circuit 57, the offset adjustment circuit 58, and the arithmetic circuit 59 will be described later with reference to FIG. 7 and the subsequent drawings. While FIG. 3 illustrates the exposure period setting circuit 56, the gain adjustment circuit 57, the offset adjustment circuit 58, and the arithmetic circuit 59 as individual circuits for clarity of explanation, these circuits may be configured as a common circuit (such as an integrated circuit (IC)).

FIG. 4 is a circuit diagram illustrating the configuration example of the detection device according to the embodiment. As illustrated in FIG. 4, a plurality of photodiodes PD1, PD2, PD3, and PD4 are coupled to the integrating circuit 46 included in the detection circuit 51 via coupling switches SSW1, SSW2, SSW3, and SSW4. The detection device 1 includes a constant current source 48. The constant current source 48 is coupled to a coupling node N1 between the photodiodes PD1, PD2, PD3, PD4 and the integrating circuit 46 via an offset switch ofs.

In the following description, the photodiodes PD1, PD2, PD3, and PD4 will each be simply referred to as the photodiode PD when need not be distinguished from one another. The coupling switches SSW1, SSW2, SSW3, and SSW4 will each be simply referred to as a coupling switch SSW when need not be distinguished from one another.

The anodes of the photodiodes PD are supplied with the second reference potential Vorg from the power supply circuit 52. The cathodes of the photodiodes PD are coupled to the detection circuit 51 via the coupling switches SSW.

Sensor capacitance Cd is coupled in parallel to the photodiode PD. The sensor capacitance Cd is capacitance generated between the upper electrode 24 and the lower electrode 23 of the photodiode PD.

The detection circuit 51 includes the integrating circuit 46 and the A/D conversion circuit 47. The integrating circuit 46 converts a variation of the photocurrent Id output from the photodiode PD into a variation of a voltage. The A/D conversion circuit 47 converts analog signals output from the integrating circuit 46 into digital signals.

The integrating circuit 46 includes the operational amplifier 45, feedback capacitance Cfb, and the reset switch rsw. The inverting input (−) of the operational amplifier 45 is coupled to the photodiodes PD via the coupling switches SSW. The photodiodes PD1, PD2, PD3, and PD4 are coupled in parallel to the inverting input (−) of the integrating circuit 46 via the coupling switches SSW1, SSW2, SSW3, and SSW4.

The first reference potential Vref is supplied from the power supply circuit 52 to the non-inverting input (+) of the operational amplifier 45. The output of the operational amplifier 45 is coupled to the A/D conversion circuit 47. An output voltage Vout of the integrating circuit 46 is a voltage value at a coupling node N2 between the operational amplifier 45 and the A/D conversion circuit 47.

The integrating circuit 46 is provided to be changeable in gain. That is, the gain of the integrating circuit 46 can be adjusted by changing the capacitance value of the feedback capacitance Cfb. The feedback capacitance Cfb may be configured as a variable capacitance element, or a plurality of different capacitance elements may be provided and configured to be switchable by a switch or the like (not illustrated). The reset switch rsw is provided to reset the electric charge of the feedback capacitance Cfb of the integrating circuit 46 during a reset period.

FIG. 5 is a timing diagram for explaining a detection method of the detection device according to the embodiment. As illustrated in FIG. 5, the detection device 1 has a measurement cycle interval adjustment period T0, an external light measurement period T1, and measurement periods T2, T3, and T4. One-cycle (1-cycle) measurement, including the measurement cycle interval adjustment period T0, the external light measurement period T1, and the measurement periods T2, T3, and T4, is repeated a plurality of times at a predetermined interval.

The measurement cycle interval adjustment period T0 is provided to adjust the measurement cycle interval for performing the one-cycle (1-cycle) measurement. During the measurement cycle interval adjustment period T0, the coupling switches SSW and the reset switch rsw are on and the offset switch ofs is off.

During the external light measurement period T1, the detection circuit 51 measures external light irradiating the photodiodes PD. During the external light measurement period T1, the first and the second light sources 61 and 62 are unlit (off), and only the external light irradiates the photodiodes PD. From time t1, the coupling switches SSW1, SSW2, SSW3, and SSW4 are sequentially turned on. During a period when the coupling switch SSW1 is on, the photocurrent Id corresponding to the electric charge stored in the sensor capacitance Cd of the photodiode PD1 flows. The photocurrent Id in the external light measurement period T1 has a current value corresponding to the intensity of the external light.

During the period when the coupling switch SSW1 is on, the reset switch rsw repeats to be turned on and off a plurality of times. In FIG. 5, the period when the coupling switch SSW1 is on includes two periods Tchk when the reset switch rsw is on. However, the number of the periods Tchk is simplified to facilitate viewing of the drawing, and three or more of the periods Tchk may be provided depending on the required measurement accuracy of the external light intensity.

During the external light measurement period T1, the offset switch ofs is on. The constant current source 48 changes the setting of an offset current Idac for each of the periods Tchk. During the period when the reset switch rsw is off, the offset current Idac having a changed current value flows from the constant current source 48. The output voltage Vout of the integrating circuit 46 changes with the offset current Idac for each of the periods Tchk. The detection circuit 51 sets the offset current Idac that makes the output voltage Vout equivalent to the first reference potential Vref, as a photocurrent Id_ol caused by the external light. That is, the detection circuit 51 sets the offset current Idac that maintains the output voltage Vout at the first reference potential Vref even when a predetermined time has elapsed after the reset switch rsw is changed from on to off, as the photocurrent Id_ol caused by the external light.

In the external light measurement period T1 in FIG. 5, the photocurrent Id_ol caused by the external light is measured for each of the photodiodes PD1, PD2, PD3, and PD4 in a time-division manner. The measured photocurrent Id_ol caused by the external light is stored in the storage circuit 54 for each of the photodiodes PD1, PD2, PD3, and PD4. However, the present disclosure is not limited to this way of measurement. In the external light measurement period T1, the coupling switches SSW1, SSW2, SSW3, and SSW4 may be turned on, and the photocurrents Id_ol caused by the external light may be simultaneously measured using the photodiodes PD1, PD2, PD3, and PD4. In this case, the average of the photocurrents Id_ol caused by the external light may be set as the photocurrent Id_ol caused by the external light for each of the photodiodes PD.

The adjustment circuit 55 (refer to FIG. 3) adjusts various parameters, such as a gain, an offset amount, and other parameters in the output voltage characteristic of the integrating circuit 46 based on the external light intensity measured in the external light measurement period T1. The light source drive circuit 53 (refer to FIG. 3) sets the light intensities of the first and the second light sources 61 and 62 based on the external light intensity measured in the external light measurement period T1.

Then, in the measurement periods T2, T3, and T4, the detection device 1 switches the first and the second light sources 61 and 62 on and off to measure the object to be detected. During the measurement period T2, the first and the second light sources 61 and 62 are off, and the object to be detected is measured using only the external light. During the measurement period T3, the first light source 61 is lit (on) and the second light source 62 is unlit (off), thus, the object to be detected being measured using the first light source 61 and the external light. In the measurement period T4, the first light source 61 is off and the second light source 62 is on, thus, the object to be detected being measured using the second light source 62 and the external light.

The measurement periods T2, T3, and T4 each include the exposure period Texp and a readout period Tread. In the following description, the measurement period T2 out of the measurement periods T2, T3, and T4 will be described. The measurement method in the measurement periods T3 and T4 is the same as in the measurement period T2. At time t2, the coupling switch SSW1 is turned on, and after a lapse of a predetermined amount of time, the coupling switch SSW1 is turned off. The effective exposure period Texp starts at the time when the coupling switch SSW1 is turned off. During the period when the light source is on, a current corresponding to a light intensity flows through the photodiode PD1. At this time, when the coupling switch SSW1 is on, the potential on the cathode side of the photodiode PD1 remains equal to the first reference potential Vref. When the coupling switch SSW1 is turned off, a current that corresponds to the intensity of the light and flows through the photodiode PD1 flows into the sensor capacitance Cd. As a result, an electric charge is stored in the sensor capacitance Cd.

After the coupling switch SSW1 is turned off, the coupling switches SSW2, SSW3, and SSW4 are sequentially controlled on and off in a time-division manner, and the effective exposure periods Texp of the photodiodes PD2, PD3, and PD4 start sequentially at different times.

During a period including the exposure period Texp, the reset switch rsw is on and the integrating circuit 46 is reset. At a time before the readout period Tread, the reset switch rsw is turned off and the integrating circuit 46 is released from the reset state.

When the coupling switch SSW1 is turned on at time t3, the readout period Tread starts, and a current corresponding to the electric charge stored in the sensor capacitance Cd flows into the feedback capacitance Cfb in the integrating circuit 46. The offset period Tdac is set to overlap the readout period Tread. In the offset period Tdac, the offset switch ofs is turned on, and the offset current Idac set by the adjustment circuit 55 (refer to FIG. 3) flows from the constant current source 48. The integrating circuit 46 converts the offset current Idac and the current flowing into the capacitance Cd during the readout period Tread into a voltage, and outputs the output voltage Vout.

After coupling the switch SSW1 is turned off, the coupling switches SSW2, SSW3, and SSW4 are sequentially turned on, and the readout periods Tread of the photodiodes PD2, PD3, and PD4 sequentially start at different times. The principle of reading out from the photodiodes PD2, PD3, and PD4 is the same as that of reading out from the photodiode PD1 described above.

The A/D conversion circuit 47 performs signal processing of the output voltage Vout output from the integrating circuit 46, and outputs the sensor value So corresponding to the photocurrent Id to the host 101. The detection circuit 51 outputs, as the sensor value So (measured value), data of the difference between the output voltage Vout of the integrating circuit 46 measured in the measurement period T3 when the first light source 61 is on and the output voltage Vout measured in the measurement period T2 when the first and the second light sources 61 and 62 are off. That is, the data of the difference obtained by subtracting the output voltage Vout based only on the external light in the measurement period T2 from the output voltage Vout based on the first light source 61 and the external light in the measurement period T3 is output as the sensor value So (measured value).

In the same way, the detection circuit 51 outputs, as the sensor value So (measured value), the data of the difference between the output voltage Vout of the integrating circuit 46 measured in the measurement period T4 when the second light source 62 is on and the output voltage Vout measured in the measurement period T2 when the first and the second light sources 61 and 62 are off. That is, the data of the difference obtained by subtracting the output voltage Vout based only on the external light in the measurement period T2 from the output voltage Vout based on the second light source 62 and the external light in the measurement period T4 is output as the sensor value So (measured value). Thus, the detection device 1 can remove the component due to the external light from the output voltage Vout in the sensor value So, and can accurately measure the object to be detected based on each of the first and the second light sources 61 and 62.

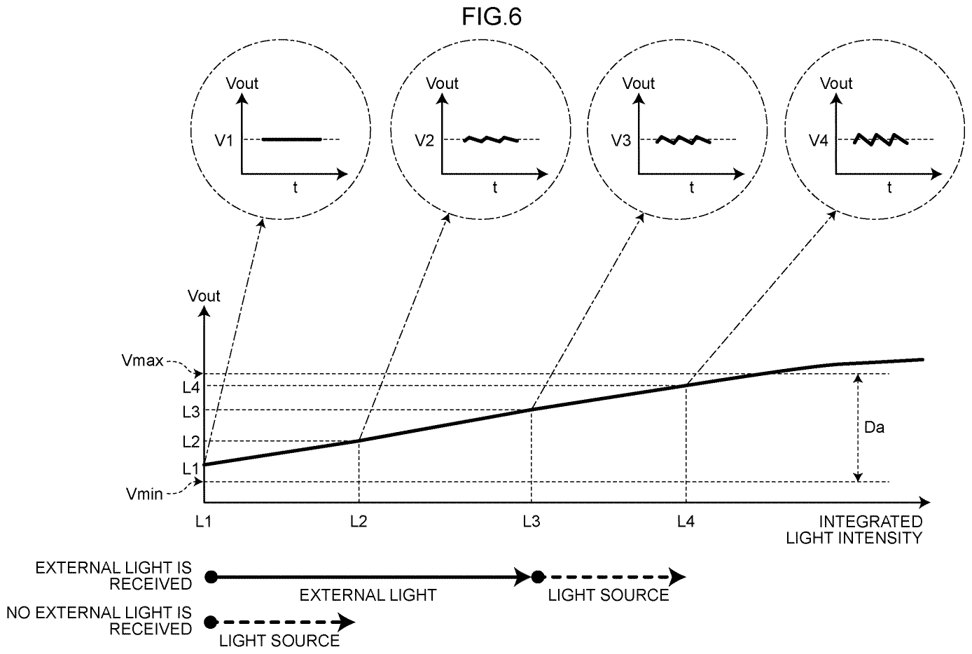

The following describes a method for adjusting the output voltage characteristics of the integrating circuit 46. FIG. 6 is an explanatory diagram illustrating a relation between an integrated light intensity and the output voltage of the integrating circuit in a detection device according to a comparative example. The horizontal axis of the graph illustrated in FIG. 6 represents the integrated light intensity, and the vertical axis represents the output voltage Vout of the integrating circuit 46. The integrated light intensity is a value obtained by integrating the light intensity emitted from the light source over the exposure period Texp. In the description with reference to FIG. 6, the exposure period Texp is assumed to have a constant length. In the following description, the first and the second light sources 61 and 62 will each be simply referred to as the “light source” when need not be distinguished from each other.

FIG. 6 also schematically illustrates a graph of a pulse wave illustrating a relation between the output voltage Vout (Vout=V1, V2, V3, or V4) and time t for each of integrated light intensities L1, L2, L3, and L4.

As illustrated in FIG. 6, the photodiode PD may be irradiated with different light intensities: from the integrated light intensity L1 when no external light is received and the light sources are off to the integrated light intensity L4 when the external light is received and the light source is on. In the comparative example, the gain and the offset are set so that a range from the output voltage Vout (=V1) based on the integrated light intensity L1 to the output voltage Vout (=V4) based on the integrated light intensity L4 falls within an effective range Da of the integrating circuit. That is, the gain and the offset of the integrating circuit are set so that an assumed minimum value of the output voltage Vout (output voltage V1) is equal to or greater than a minimum value Vmin of the effective range Da, and an assumed maximum value of the output voltage Vout (output voltage V4) is equal to or less than a maximum value Vmax of the effective range Da. Therefore, the amplitude of the output voltage Vout output from the integrating circuit 46 is measured to be smaller, which may reduce the detection accuracy.

FIG. 7 is a flowchart illustrating a method for adjusting the output voltage characteristics indicating the relation between the integrated light intensity and the output voltage of the integrating circuit in the detection device according to the embodiment. As illustrated in FIG. 7, the detection device 1 measures the photocurrent Id_ol caused by the external light (Step ST1). The measurement of the photocurrent Id_ol caused by the external light is the same as the method described for the external light measurement period T1 in FIG. 5, and will not be described again. The photocurrent Id_ol caused by the external light may be measured at intervals of a predetermined measurement period in which a plurality of cycles of measurement are performed. The present disclosure is not limited to this method. The photocurrent Id_ol caused by the external light may be measured for each cycle or at a predetermined timing (for example, at power-on of the detection device 1).

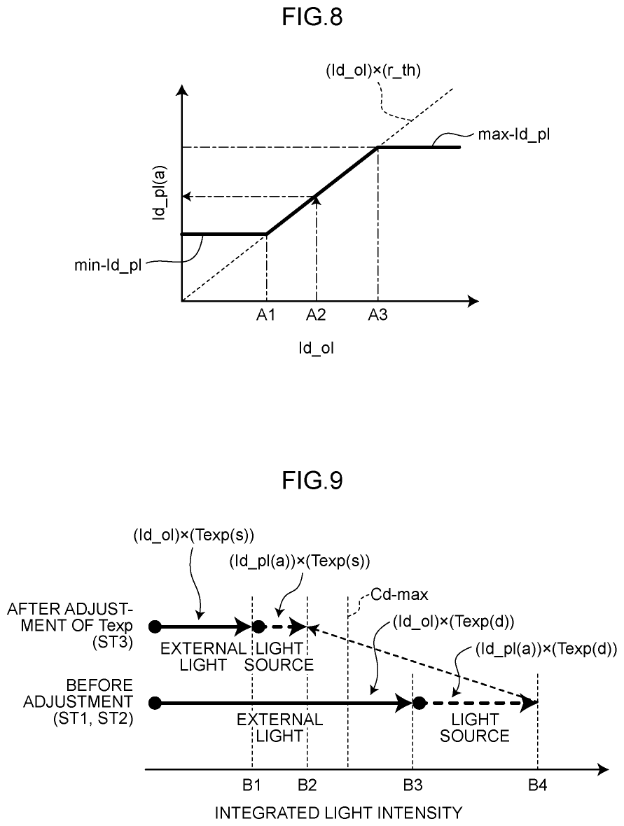

The detection device 1 then calculates a virtual photocurrent Id_pl(a) caused by the light source (Step ST2). FIG. 8 is a graph for explaining a method for calculating the virtual photocurrent caused by the light source at Step ST2 in FIG. 7. The horizontal axis of the graph illustrated in FIG. 8 represents the photocurrent Id_ol caused by the external light, and the vertical axis represents the virtual photocurrent Id_pl(a) caused by the light source.

In FIG. 8, a coefficient r_th represents the ratio of the light intensity of the light source to the light intensity of the external light. The coefficient r_th is set in advance based on past measurement results and other factors and stored in the storage circuit 54. The coefficient r_th is approximately 0.3, for example. The coefficient r_th is set so that the photocurrent Id_pl caused by the light source can be measured in the detection circuit 51 even in the presence of the external light.

As illustrated in FIG. 8, the virtual photocurrent Id_pl(a) caused by the light source is calculated by multiplying the photocurrent Id_ol caused by the external light measured at Step ST1 (for example, the photocurrent Id_ol caused by the external light=A2) by the coefficient r_th based on the Expression (1) given below.

$$Id\_pl ( a ) = ( Id\_ol ) \times ( r\_th ) \qquad ( 1 )$$

If the photocurrent Id_ol caused by the external light is A1 to A3, the virtual photocurrent Id_pl(a) caused by the light source is calculated based on Expression (1). If the photocurrent Id_ol caused by the external light is less than A1, the virtual photocurrent Id_pl(a) caused by the light source is set to a minimum photocurrent min-Id_pl. If the photocurrent Id_ol caused by the external light is greater than A3, the virtual photocurrent Id_pl(a) caused by the light source is set to a maximum photocurrent max-Id_pl.

The minimum photocurrent min-Id_pl is the photocurrent Id_pl caused by the light source that can achieve a predetermined amplitude of the output voltage Vout in the detection circuit 51 in the absence of the external light. The maximum photocurrent max-Id_pl is the photocurrent Id_pl caused by the light source that flows through the photodiode PD when the maximum current (rated current) is applied to each of the first and the second light sources 61 and 62. The minimum photocurrent min-Id_pl and the maximum photocurrent max-Id_pl are set in advance and stored in the storage circuit 54.

The detection device 1 then calculates the exposure period Texp (Step ST3). FIG. 9 is an explanatory diagram for explaining a method for calculating the exposure period at Step ST3 in FIG. 7. In FIG. 9, a threshold Cd-max is the maximum value of the integrated light intensity corresponding to the saturation capacitance in the sensor capacitance Cd of the photodiode PD.

As illustrated in FIG. 9, if the photocurrent Id_ol caused by the external light and the virtual photocurrent Id_pl(a) caused by the light source flow over an exposure period Texp(d) before adjustment, an integrated light intensity B4 may exceed the threshold Cd-max. An integrated light intensity B3 of the photocurrent Id_ol caused by the external light is expressed as B3=(Id_ol)×(Texp(d)). The integrated light intensity B4 is expressed as B4=(Id_ol)×(Texp(d))+(Id_pl(a))×(Texp(d)).

The exposure period setting circuit 56 (refer to FIG. 3) sets an adjusted exposure period Texp(s) based on the photocurrent Id_ol caused by the external light obtained at Steps ST1 and ST2 and the virtual photocurrent Id_pl(a) caused by the light source so that the sensor capacitance Cd does not saturate. More specifically, the exposure period setting circuit 56 (refer to FIG. 3) determines the adjusted exposure period Texp(s) based on Expression (2) given below.

$$Texp ( s ) = ( Cd \times ( Vref - Vorg ) \times r\_margin1 ) / ( ( Id\_ol ) + ( Id\_pl ( a ) ) ) + Tread \qquad ( 2 )$$

r_margin1 in Expression (2) is a margin of the sensor capacitance Cd with respect to the saturation capacitance. The margin r_margin1 is a preset parameter and is stored in the storage circuit 54. For example, the margin r_margin1 is set to approximately 0.7.

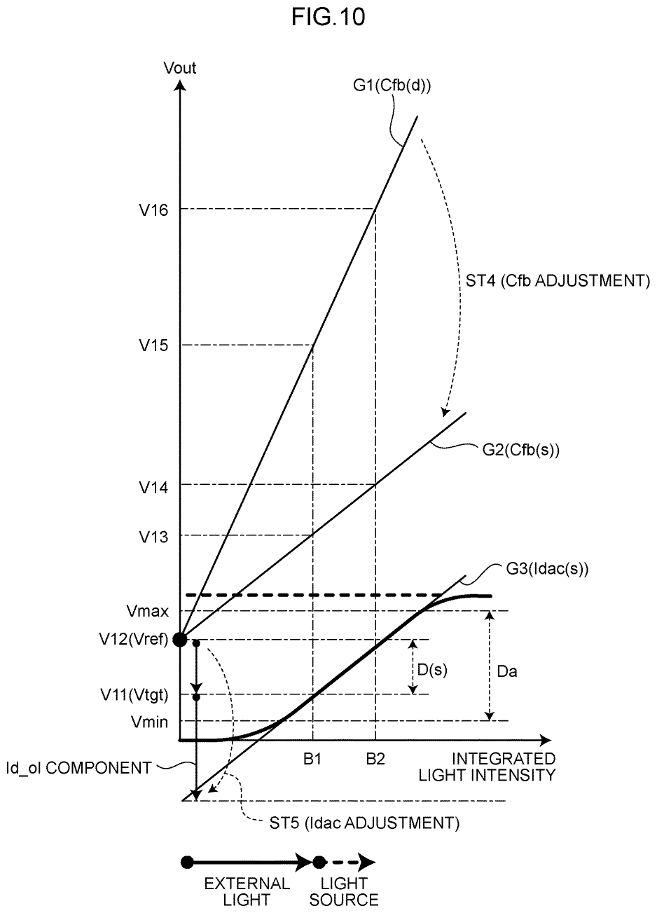

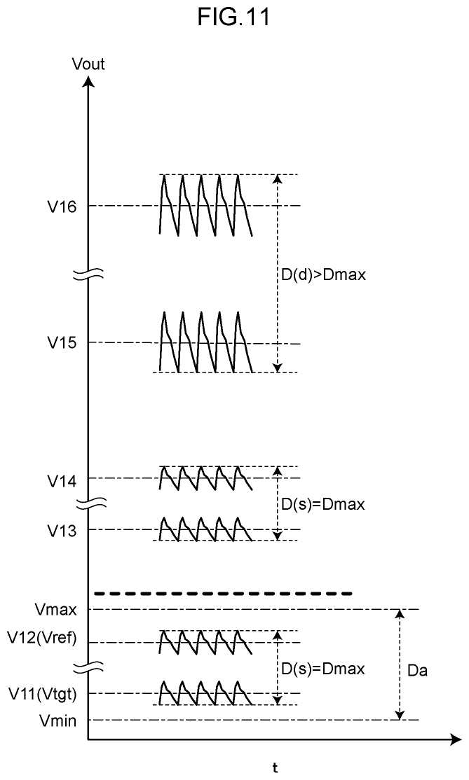

The detection device 1 then determines the gain of the integrating circuit 46 (Step ST4). FIG. 10 is a graph for explaining a method for adjusting the gain and the offset amount at Steps ST4 and ST5 in FIG. 7. FIG. 11 is a graph for explaining a waveform and a measurement range of each output voltage in FIG. 10.

The horizontal axis of the graph illustrated in FIG. 10 represents the integrated light intensity, and the vertical axis represents the output voltage Vout of the integrating circuit 46. The output voltage characteristics G1, G2, and G3 illustrated in FIG. 10 each indicates a relation between the intensity of the light irradiating the photodiode PD and the output voltage Vout of the integrating circuit 46. Integrated light intensities B1 and B2 in FIG. 10 are integrated light intensities corresponding to the adjusted exposure period Texp(s) obtained at Step ST3.

In FIG. 11, respective waveforms (pulse waves) of the output voltages Vout (=V15 and V16) of the output voltage characteristics G1, the output voltages Vout (=V13 and V14) of the output voltage characteristics G2, and the output voltages Vout (=V11 and V12) of the output voltage characteristics G3 in FIG. 10 are schematically illustrated on the same time axis.

As illustrated in FIG. 10, in the output voltage characteristics G1 corresponding to feedback capacitance Cfb(d) before adjustment, the output voltage Vout of the integrating circuit 46 corresponding to the integrated light intensity B1 when the light source is off (external light alone) is Vout=V15. The output voltage Vout of the integrating circuit 46 corresponding to the integrated light intensity B2 when the light source is on (external light and light source) is Vout=V16.

As illustrated in FIG. 11, a measurement range D(d) before adjustment is represented by the difference between the output voltage Vout (=V16) of the integrated light intensity B2 (when light source is on) and the output voltage Vout (=V15) of the integrated light intensity B1 (when light source is off). In more detail, the measurement range D(d) before adjustment is the difference between the maximum level in the amplitude of the output voltage V16 and the minimum level in the amplitude of the output voltage V15.

The measurement range D(d) before adjustment is larger than an effective range Dmax. The effective range Dmax is determined by multiplying the effective range Da (refer to FIG. 6) of the integrating circuit 46 by the margin r_margin2 (Dmax=Da×(r_margin2)). The margin r_margin2 is a parameter set in advance taking into account a temporal variation of a direct-current (DC) component of the output voltage Vout, and is stored in the storage circuit 54. For example, the margin r_margin2 is set to approximately 0.6.

At Step ST4, the integrating circuit 46 sets the gain smaller as the photocurrent Id_ol (light intensity of external light) caused by the external light measured at Step ST1 with the light source off is larger. In more detail, the gain adjustment circuit 57 (refer to FIG. 3) is configured with the feedback capacitance Cfb of the integrating circuit 46 and is capable of adjusting the gain of the integrating circuit 46 by changing the capacitance value of the feedback capacitance Cfb.

As illustrated in FIGS. 10 and 11, the gain adjustment circuit 57 (refer to FIG. 3) adjusts the feedback capacitance Cfb so that the measurement range D(d) before adjustment falls within the effective range Dmax. Specifically, the gain adjustment circuit 57 (refer to FIG. 3) determines an adjusted feedback capacitance Cfb(s) based on Expression (3) given below.

$$Cfb ( s ) = ( Id\_pl ( a ) + ( Id\_ol + Id\_pl ( a ) ) \times ( r\_ac ) ) \times Texp ( s ) / Dmax \qquad ( 3 )$$

In Expression (3), a coefficient r_ac is the maximum value of the ratio of the alternating-current component (ac) to the direct-current (dc) of the output voltage Vout (i.e., ac/dc). The coefficient r_ac is set to approximately 0.05 (5%), for example.

As illustrated in FIG. 10, the output voltage characteristics G2 after gain adjustment has a smaller slope than the output voltage characteristics G1 before adjustment. In the output voltage characteristics G2 after gain adjustment, the output voltage Vout of the integrating circuit 46 corresponding to the integrated light intensity B1 is Vout=V13. The output voltage Vout of the integrating circuit 46 corresponding to the integrated light intensity B2 is Vout=V14. The difference between the output voltage V14 and the output voltage V13 (V14−V13) is smaller than the difference between the output voltage V16 and the output voltage V15 (V16−V15) in the output voltage characteristics G1 before adjustment.

As illustrated in FIG. 11, an adjusted measurement range D(s) is the difference between the maximum level in the amplitude of the output voltage V14 at the integrated light intensity B2 and the minimum level in the amplitude of the output voltage V13 at the integrated light intensity B1. The adjusted measurement range D(s) is set equal to the effective range Dmax.

The detection device 1 then determines the offset amount of the integrating circuit 46 (Step ST5). The offset amount of the output voltage characteristics G2 of the integrating circuit 46 can be adjusted by changing the offset current Idac of the constant current source 48 (refer to FIG. 4). In other words, the offset adjustment circuit 58 (refer to FIG. 3) includes the constant current source 48 coupled to the coupling node N1 between the photodiodes PD and the detection circuit 51.

As illustrated in FIG. 10, the offset adjustment circuit 58 determines an adjusted offset current Idac(s) so that the output voltage Vout of the integrating circuit 46 corresponding to the integrated light intensity B1 (when light source is off) becomes a target voltage Vtgt for the output voltage characteristics G2 after gain adjustment obtained at Step ST4. More specifically, the offset adjustment circuit 58 determines the offset current Idac(s) based on the component (voltage value) of the photocurrent Id_ol caused by the external light and the difference between the first reference potential Vref and the target voltage Vtgt. The offset adjustment circuit 58 shifts the output voltage characteristics G2 after gain adjustment obtained at Step ST4 more toward the lower voltage side, as the photocurrent Id_ol caused by the external light (light intensity of external light) is larger.

The adjusted offset current Idac(s) is expressed as Expression (4) given below. In Expression (4), Tdac is the offset period Tdac illustrated in FIG. 5.

$$Idac ( s ) = ( ( Id\_ol ) \times ( Texp ( s ) + Cfb ( s ) \times ( Vtgt - Vref ) ) / Tdac \qquad ( 4 )$$

As illustrated in FIGS. 10 and 11, in the output voltage characteristics G3 after offset adjustment, the output voltage Vout of the integrating circuit 46 corresponding to the integrated light intensity B1 is shifted to Vout=V11. The output voltage V11 is a voltage value equal to the target voltage Vtgt. The output voltage Vout of the integrating circuit 46 corresponding to the integrated light intensity B2 is shifted to Vout=V12. The output voltage V12 is a voltage value equal to the first reference potential Vref. The difference between the output voltage V12 and the output voltage V11 (V12−V11) is ideally equal to the difference between the output voltage V14 and the output voltage V13 (V14−V13) in the output voltage characteristics G2.

The measurement range D(s) in the output voltage characteristics G3 after offset adjustment is located within the effective range Da of the integrating circuit 46. As illustrated in FIG. 11, the minimum level in the amplitude of the output voltage V11 of the integrating circuit 46 corresponding to the integrated light intensity B1 (when light source is off) is equal to or greater than the minimum value Vmin of the effective range Da. The maximum level in the amplitude of the output voltage V12 of the integrating circuit 46 corresponding to the integrated light intensity B2 (when light source is on) is equal to or less than the maximum value Vmax of the effective range Da.

The detection device 1 then measures the object to be detected under the conditions of the gain and the offset amount of the integrating circuit 46 and the exposure period Texp in the detection circuit 51 that have been determined at Steps ST3 to ST5 (Step ST6). In the measurement of the object to be detected at Step ST6, the light intensities of the first and the second light sources 61 and 62 are determined according to the photocurrent Id_ol caused by the external light.

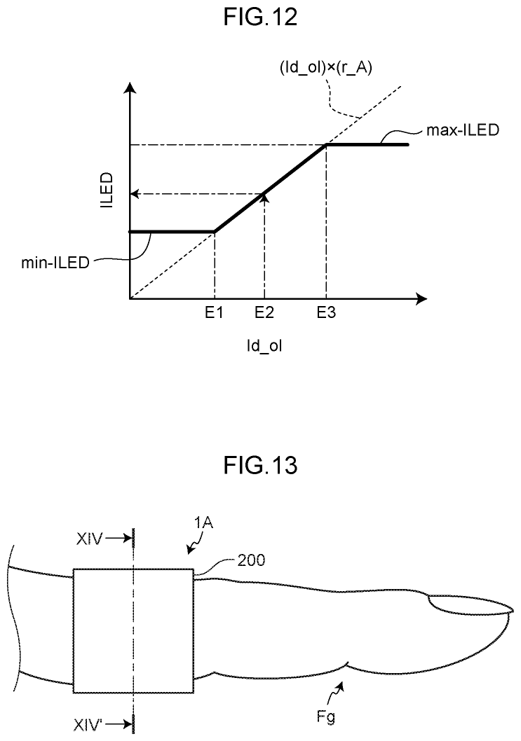

FIG. 12 is a graph for explaining a method for calculating a drive current to be supplied to the light sources. The horizontal axis of the graph illustrated in FIG. 12 represents the photocurrent Id_ol caused by the external light, and the vertical axis represents a light source drive current ILED supplied to the first and the second light sources 61 and 62.

As illustrated in FIG. 12, the light source drive current ILED is calculated by multiplying the photocurrent Id_ol caused by the external light measured at Step ST1 (for example, the photocurrent Id_ol caused by the external light is E2) by a coefficient r_A based on Expression (5) given below. In FIG. 12, the coefficient r_A represents the ratio of the light intensity of the light sources (light source drive current ILED) to the light intensity of the external light. The coefficient r_A is set in advance and stored in the storage circuit 54.

$$ILED = ( Id\_ol ) \times ( r\_A ) \qquad ( 5 )$$

As given in Expression (5), the light source drive current ILED increases as the light intensity of the external light increases. That is, the light intensities of the first and the second light sources 61 and 62 increase as the light intensity of the external light increases. The light source drive current ILED decreases as the light intensity of the external light decreases, or in the absence of the external light. That is, the light intensities of the first and the second light sources 61 and 62 decrease as the light intensity of the external light decreases. This configuration allows the detection device 1 to reduce the power consumption of the first and the second light sources 61 and 62 compared with a case where the first and the second light sources 61 and 62 emit light at fixed light intensities regardless of the light intensity of the external light.

In more detail, the light source drive current ILED is calculated based on Expression (5) within a range where the photocurrent Id_ol caused by the external light is E1 to E3. If the photocurrent Id_ol caused by the external light is less than E1, the light source drive current ILED is set to a minimum value min-ILED. If the photocurrent Id_ol caused by the external light is greater than E3, the light source drive current ILED is set to a maximum value max-ILED.

The minimum value min-ILED of the light source drive current ILED is a minimum current value set in advance correspondingly to the photocurrent Id_pl caused by the light source that can be measured by the detection circuit 51 in the absence of the external light (for example, the minimum photocurrent min-Id_pl (refer to FIG. 8)). The maximum value max-ILED of the light source drive current ILED is the maximum current value (rated current) that can flow to each of the first and the second light sources 61 and 62.

In the measurement periods T3 and T4 (refer to FIG. 5), the light source drive circuit 53 supplies the light source drive current ILED determined based on FIG. 12 and Expression (5) to the first and the second light sources 61 and 62. The photodiode PD is irradiated with the external light and the light from the first and the second light sources 61 and 62 corresponding to the light source drive current ILED set at Step ST6.

In the measurement periods T3 and T4 (refer to FIG. 5), the coupling switch SSW is controlled on and off based on the adjusted exposure period Texp(s) set at Step ST3. As a result, an electric charge corresponding to the adjusted exposure period Texp(s) is stored in the sensor capacitance Cd. In the readout period Tread, the integrating circuit 46 included in the detection circuit 51 measures the photocurrent Id_ol caused by the external light and the photocurrent Id_pl caused by the light source with the gain and the offset amount determined at Steps ST4 and ST5.

Thus, the detection device 1 of the present embodiment determines the exposure period Texp, and the gain and the offset amount of the integrating circuit 46 based on the external light intensity, thereby appropriately adjusting the output voltage characteristics of the integrating circuit 46 compared with the comparative example described above. Therefore, regardless of the intensity of the external light, the amplitude of the output voltage Vout can be measured in the effective range Da of the integrating circuit 46, and the effective range Da of the integrating circuit 46 can be used widely. Thus, the detection device 1 can improve the detection accuracy.

Since the detection device 1 of the present embodiment can measure the large amplitude of the output voltage Vout regardless of the intensity of the external light, the need for the signal processing of the output voltage Vout such as processing to increase the number of bits in the A/D conversion circuit 47 can be reduced, compared with the comparative example described above. As a result, the detection device 1 of the present embodiment can reduce the generation of noise caused by the signal processing.

The method for adjusting the various parameters described with reference to FIGS. 7 to 12 is merely exemplary and can be changed as appropriate. For example, the values of the coefficient r_th, the coefficient r_A, and the margin r_margin1, the margin r_margin2, and other parameters are only schematic, and not limited thereto. Alternatively, the exposure period Texp, and the gain and the offset amount of the integrating circuit 46 may be determined without using the margin r_margin1 and the margin r_margin2.

### Modification

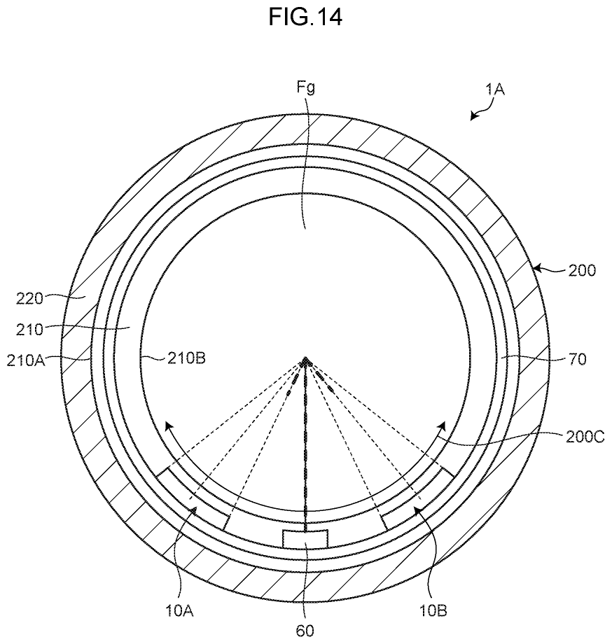

FIG. 13 is a schematic view illustrating an exemplary external appearance when a finger accommodated inside a detection device according to a modification of the embodiment is viewed from a lateral side of a housing. FIG. 14 is a sectional view taken along XIV-XIV′ in FIG. 13.

As illustrated in FIG. 13, a detection device 1A according to the modification can be applied to a ring-type device that can be worn on and removed from a human body. The detection device 1A is worn on a finger Fg of the human body. Examples of the finger Fg include a thumb, an index finger, a middle finger, a ring finger, and a little finger. The human body is a person to be authenticated whose identity is to be verified by the detection device 1A. The detection device 1A can detect biometric information on a living body from the finger Fg wearing the detection device 1A. The finger Fg is an example of a measurement target. The measurement target is the living body or a part of the living body, and is an object to be measured. The detection device 1A is formed as a finger ring or a wristband so as to be easily carriable by a user. In the following description, the detection device 1A is assumed to be used as a finger ring.

As illustrated in FIG. 14, the detection device 1A includes a housing 200, a light source 60, a first optical sensor 10A, a second optical sensor 10B, and a flexible printed circuit board 70. The detection device 1A includes a battery (not illustrated) in the housing 200 and is operated by power of the battery.

The housing 200 is a mounting member that is formed in a ring shape (annular shape) wearable on the finger Fg, and is worn on the living body. In the example illustrated in FIG. 14, the housing 200 includes a first housing 210 and a second housing 220. The first housing 210 is integrated with the second housing 220 to form the housing 200 into the ring shape. The first housing 210 is a member that contacts the human body on which the housing 200 is worn. The first housing 210 accommodates therein the light source 60, the first optical sensor 10A, the second optical sensor 10B, and other components. The first housing 210 is formed into a ring shape using a housing material, such as a light-transmitting synthetic resin or silicon. The second housing 220 has a surface of the housing 200 that covers an outer peripheral surface 210A of the first housing 210. The second housing 220 is formed into a ring shape using a member of, for example, a metal or a non-light-transmitting synthetic resin. The housing 200 accommodates, in the first housing 210, the flexible printed circuit board 70 on which the light source 60, the first optical sensor 10A, the second optical sensor 10B, and other components are mounted. The flexible printed circuit board 70 is accommodated in the housing 200, for example, by forming the housing 200 by filling the periphery of the flexible printed circuit board 70 formed into a ring shape with a filling member in a mold.

In the present modification, each of the first and the second optical sensors 10A and 10B is configured with the photodiode PD described above. The first and the second optical sensors 10A and 10B are provided so as to interpose the light source 60 therebetween in a circumferential direction 200C. That is, in the detection device 1A, the first optical sensor 10A, the light source 60, and the second optical sensor 10B are arranged in this order in the circumferential direction 200C. The first and the second optical sensors 10A and 10B are provided so as to interpose the light source 60 therebetween in the circumferential direction 200C, thereby allowing light emitted by the light source 60 to be detected over a wide area of the housing 200.

The same method for adjusting the exposure period Texp, and the gain and the offset amount of the integrating circuit 46 of the detection device 1 according to the embodiment can be applied also to the detection device 1A according to the modification. The configuration illustrated in FIGS. 13 and 14 is merely exemplary, and can be changed as appropriate. For example, FIG. 14 illustrates one light source 60, but the present disclosure is not limited to this configuration. A plurality of the light sources 60 (the first light source 61 and the second light source 62) may be included so as to be capable of emitting near-infrared, red, and green light rays.

While the preferred embodiment of the present disclosure has been described above, the present disclosure is not limited to the embodiment described above. The content disclosed in the embodiment is merely an example, and can be variously modified within the scope not departing from the gist of the present disclosure. Any modifications appropriately made within the scope not departing from the gist of the present disclosure also naturally belong to the technical scope of the present disclosure. At least one of various omissions, substitutions, and changes of the components can be made without departing from the gist of the embodiments and the modifications thereof described above.

What is claimed is:

1. A detection device comprising:
an optical sensor;
a light source configured to emit light to the optical sensor; and
a detection circuit comprising an integrating circuit coupled to the optical sensor, wherein
the integrating circuit is provided so as to be changeable in gain corresponding to a slope of output voltage characteristics representing a relation between a light intensity irradiating the optical sensor and an output voltage of the integrating circuit,
the optical sensor is configured to measure the light intensity both when the light source is off and when the light source is on, and
the integrating circuit is configured to be reduced in the gain as a light intensity of external light measured with the light source off increases.

2. The detection device according to claim 1, wherein the detection circuit is configured to output data of a difference between the output voltage of the integrating circuit when the light source is off and the output voltage of the integrating circuit when the light source is on, as a measured value.

3. The detection device according to claim 1, wherein a light intensity of the light source is increased as the light intensity of the external light increases.

4. The detection device according to claim 1, comprising an offset adjustment circuit, wherein
the offset adjustment circuit is configured to shift the output voltage characteristics more toward a lower voltage side as the light intensity of the external light is larger.

5. The detection device according to claim 4, wherein the offset adjustment circuit comprises a constant current source coupled to a coupling node between the optical sensor and the detection circuit.

6. The detection device according to claim 1, comprising a gain adjustment circuit, wherein
the gain adjustment circuit comprises feedback capacitance included in the integrating circuit, and is configured to adjust the gain of the integrating circuit by changing a capacitance value of the feedback capacitance.

7. The detection device according to claim 1, wherein the optical sensor is an organic photodiode (OPD).
